(19) United States
(12) Patent Application Publication
(10) Pub. No.: US 2024/0295571 A1
(43) Pub. Date: Sep. 5, 2024

(54) OPIOID DETECTION KIT AND METHOD THEREOF

(71) Applicant: Summit Glove Inc., Minerva, OH (US)

(72) Inventor: James L. Hull, Malvern, OH (US)

(73) Assignee: Summit Glove Inc., Minerva, OH (US)

(21) Appl. No.: 18/591,126

(22) Filed: Feb. 29, 2024

Related U.S. Application Data
(60) Provisional application No. 63/488,345, filed on Mar. 3, 2023.
(60) Provisional application No. 63/512,792, filed on Jul. 10, 2023.

(51) Int. Cl. G01N 33/94

(52) U.S. Cl. CPC G01N 33/94

(57) ABSTRACT

A self-contained substance detection kit has a container defining a first compartment and a second compartment. There is a wetting agent in the first compartment. There is at least one substrate in the second compartment. The substrate carries a dye that produces a visible color change upon exposure to an opioid or other amine-containing drug. There is a breakable seal between the first compartment and the second compartment. The breakable seal ruptures in response to applying pressure to the first compartment, wherein when the breakable seal is broken or rupture, an aperture is defined that establishes open fluid communication between the first compartment and the second compartment to allow the wetting agent to flow into the second compartment and wet the at least one substrate.

### CROSS REFERENCE TO RELATED APPLICATIONS

This application claims the benefit of U.S. Provisional Application Ser. No. 63/488,345 filed Mar. 3, 2023, the entirety of which is incorporated herein.

This application claims the benefit of U.S. Provisional Application Ser. No. 63/512,792 filed Jul. 10, 2023, the entirety of which is incorporated herein.

### TECHNICAL FIELD

This disclosure is directed to packages and kits that contain a material that changes color to indicate the presence of a substance.

### BACKGROUND ART

U.S. Patent Application Publication No. 2021/0302446 indicates that according to the Center for Disease Control (CDC), over one hundred people died every day of 2016 from opioid overdose, accounting for more than 42,000 opioid related deaths in the U.S. that year alone. The annual increase in opioid addiction has been highest in Ohio and New Hampshire, with opioid overdoses increasing 70% from July 2016 to September 2017. First responders to the scenes of these overdoses must be extremely cautious of inadvertent exposure to these potent opioid substances. For instance, carfentanyl has a lethal dose of about 2 milligrams (mg), and thus, particles that inadvertently come in contact with law enforcement, emergency medical services, firefighters, and other first responders could be fatal. In spite of the significant risk, there is currently no quick, cost-effective and rapid technology that enables a first responder to make a confident determination of the hazardous nature of a suspicious drug. Existing technologies for detecting the presence of opioids include Raman/IR detectors and ‘spot test’ kits. Raman and IR detectors are very accurate and can identify samples from a safe distance and without opening the packaging. However, the high cost of these hand-held detectors (˜$20,000) prevents their deployment to every first responder.

Drug detection tests, sometimes referred to as spot tests, have been designed to differentiate between various opioids, amphetamines and other illicit drugs. These tests have been developed, modified and optimized to identify closely related classes of illicit drugs and are very useful in the prosecution of a crime. The Marquis reagent, for example, may be employed for the detection of morphine and other alkaloids, which show a red-violet color. This test has also been used as a general screening test for many drugs including various types of amphetamines. Several other tests such as Simon's reagent (for secondary amines), Mecke (for opium alkaloids), Liebermann's (for phenols), Fast blue (for cannabinoids), and Zwikker (for barbiturates) have been developed. These reagents are designed to react with functional groups of the drugs to form color compounds.

Currently, the most common drug detection tests come in the form of kits that include pouches that contain various ampules. The user must pick up and transfer the suspected drug to the pouch and break the various ampules to initiate a reaction. The solution changes color and the color itself can be used to identify the drug. These methods can be highly accurate and reproducible. However, these methods require the user to pick up the drug, and this puts the user in danger of exposure that may be potentially fatal in case of highly lethal drugs like carfentanyl. This causes undue safety issues to the first responder who has to carry out the test on-site under high stress conditions. Other drug detection products use a similar technology along with an applicator or sample collection surface.

Current wipe-based products use a cobalt thiocyanate dye to indicate, by color change, contact with cocaine. Unfortunately, cobalt thiocyanate is toxic, and these wipe products cannot be used on surfaces that may come into human contact.

Other products that are available include test pens. The technology is based on antigen-enzyme binding. The small applicator tip of the pen also restricts detection of drugs on large surfaces. Also, aerosolized sprays are also available that that are also based upon antigen-enzyme binding technology. However, products that rely upon antigen-enzyme binding technology are relatively expensive.

There exists a need for a process and affordable product that allows for the rapid detection of dangerous opioids. One such process is taught by US Patent Application Publication Number 2021/0302446 (“the '446 Publication”). The '446 Publication has shown great promise for its results in the laboratory. However, implementing it for use in the field has proved difficult. Particularly, the application of the wetting agent in the field can be difficult.

### SUMMARY OF THE INVENTION

What is needed is a self-contained kit for a substance detection, wherein all necessary components of the kit are provided in a single package so the first responder may effectuate the rapid detection of the substance as evidenced by a change in color on a substrate without needing to collect all the components from separate sources or locations.

In one aspect, an exemplary embodiment of the present disclosure may provide a self-contained substance detection kit comprising: a flexible plastic container defining a first compartment and a second compartment; a wetting agent in the first compartment, wherein the wetting agent is an aqueous wetting agent; at least one substrate in the second compartment, wherein the at least one substrate carries a dye that produces a visible color change upon exposure to an opioid or other amine-containing drug; and a breakable seal between the first compartment and the second compartment, wherein the breakable seal is breakable in response to applying pressure to the first compartment, wherein when the breakable seal is broken, an aperture is defined that establishes open fluid communication between the first compartment and the second compartment to allow the wetting agent to flow into the second compartment and wet the at least one substrate. This exemplary embodiment or another exemplary embodiment may further provide that the dye is selected from the group consisting of malachite green oxalate, brilliant green, eosin yellowish, erythrosine b, methyl green, methyl violet, picric acid, cresol red, crystal violet, m-cresol purple, thymol blue, bromocresol green, cresol red, p-xylenol blue, eosin bluish, quinaldine red, 2,4-dinitro phenol, 4-(dimethylamino) azobenzol, bromochlorophenol blue, bromophenol blue, congo red, methyl orange, 1-naphtholphthalein, m-cresol purple, thymol blue, p-xylenol blue, phenolphthalein, thymolphthalein, alkali blue, alizarin yellow gg, indigo carmine, epsilon blue, and titan yellow. This exemplary embodiment or another exemplary embodiment may further provide that the target analyte is selected from the group consisting of amine-containing drugs. This exemplary embodiment or another exemplary embodiment may further provide that the target analyte is selected from the group consisting of morphine, hydrocodone, oxycodone, fentanyl, carfentanyl, naloxone, endorphins, heroin, codeine, cocaine, and mixtures and derivatives thereof. This exemplary embodiment or another exemplary embodiment may further provide that the substrate comprises a film, a woven fabric, a nonwoven fabric, a cellulose tissue, a paper towel, a coform material, an airlaid material, a polyester amide polymer having pendant functional groups, or a bonded-carded web. This exemplary embodiment or another exemplary embodiment may further provide that the substrate comprises natural fibers, synthetic fibers, or combinations thereof. This exemplary embodiment or another exemplary embodiment may further provide that the substrate comprises fibers selected from the group consisting of wood pulp, cotton, bamboo, hemp, polyolefin, polyester, polyamide, polylactic acid, rayon, lyocell, and combinations thereof. This exemplary embodiment or another exemplary embodiment may further provide that the substrate is in the form of a wipe, a swab, or a glove. This exemplary embodiment or another exemplary embodiment may further provide a color enhancer and a catalyst. This exemplary embodiment or another exemplary embodiment may further provide that the device is an airtight package. This exemplary embodiment or another exemplary embodiment may further provide that the aqueous wetting agent comprises at least one of water and a C1-6 alcohol. This exemplary embodiment or another exemplary embodiment may further provide that the at least one substrate defines a glove within the second compartment. This exemplary embodiment or another exemplary embodiment may further provide that the glove is symmetric and adapted to allow either face of the glove.

In yet another aspect, another exemplary embodiment of the present disclosure may provide a method for detecting the presence of a substance, the method comprising: providing a self-contained substance detection kit including a flexible plastic container defining a first compartment and a second compartment, the first compartment containing a wetting agent, wherein the wetting agent is an aqueous wetting agent, and the second compartment containing at least one substrate, wherein the at least one substrate carries a dye that produces a visible color change upon exposure to an opioid or other amine-containing drug; breaking a breakable seal disposed between the first compartment and the second compartment, such that the wetting agent flows into the second compartment; wetting the at least one substrate; opening the flexible plastic container to remove the at least one substrate; contacting the at least one substrate with a surface; determining whether the dye has changed color, wherein a change in color is indicative of a presence of the opioid or other amine-containing drug. This exemplary embodiment or another exemplary embodiment may further provide for visually inspecting the at least one substrate after the step of contacting, to determine if any portion of the at least one substrate has changed color. This exemplary embodiment or another exemplary embodiment may further provide that at least a portion of the drug detection device undergoes a visible color change within about 30 seconds of contact with a target analyte. This exemplary embodiment or another exemplary embodiment may further provide that at least a portion of the drug detection device undergoes a visible color change within about 10 seconds of contact with a target analyte. This exemplary embodiment or another exemplary embodiment may further provide that at least a portion of the drug detection device undergoes a visible color change within about 5 seconds of contact with a target analyte.

In yet another aspect, an exemplary embodiment of the present disclosure provides a substance detection kit comprising a flexible plastic container. The flexible plastic container defines a first compartment and a second compartment. The first compartment contains a wetting agent. The second compartment contains at least one substrate. A breakable seal may be disposed between the first compartment and the second compartment. The breakable seal is breakable to allow the wetting agent fluid to flow into the second compartment in response to rupture of the breakable seal. The at least one substrate may comprises a liquid-absorbing material that absorbs the wetting agent fluid flowing into the second compartment.

### BRIEF DESCRIPTION OF THE DRAWINGS

Sample embodiments of the present disclosure are set forth in the following description, are shown in the drawings and are particularly and distinctly pointed out and set forth in the appended claims.

FIG. 1 is a perspective view of an exemplary self-contained substance detection kit according to one embodiment of the present disclosure.

FIG. 2 is a top plan view of the exemplary self-contained substance detection kit according to one embodiment of the present disclosure with a breakable seal having been ruptured or otherwise broken.

FIG. 3 is a perspective view of the exemplary self-contained substance detection kit according to one embodiment of the present disclosure with a breakable seal having been ruptured or otherwise broken and one compartment being opened to remove a substrate having been wetted by a wetting agent.

FIG. 4A is a view depicting one alternative form or instantiation of the substrate in the shape of a glove.

FIG. 4B is a view depicting another alternative form or instantiation of the substrate in the shape of a symmetric glove.

FIG. 4C is a view depicting another alternative form or instantiation of the substrate in the shape of a mitten.

FIG. 5A is a side elevation cross section view of another exemplary self-contained substance detection kit according to another embodiment of the present disclosure.

FIG. 5B is a top plan view of another exemplary self-contained substance detection kit according to another embodiment of the present disclosure.

Similar numbers refer to similar parts throughout the drawings.

### DETAILED DESCRIPTION

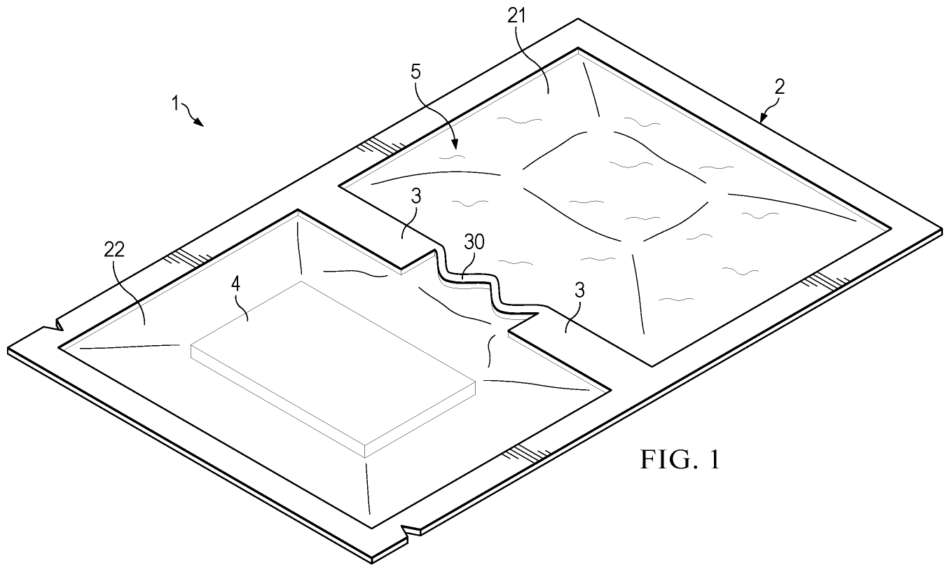

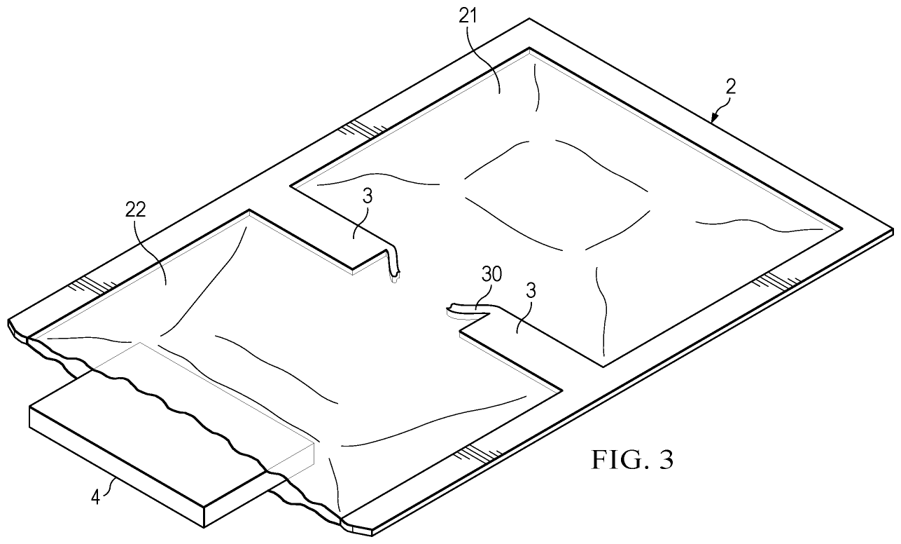

As illustrated in FIGS. 1-3, in some embodiments, a substance detection kit 1 that comprises a flexible plastic container 2. The flexible plastic container 2 defines a first compartment 21 and a second compartment 22. The first compartment 21 contains a fluid wetting agent 5. In some embodiments, the fluid wetting agent 5 is non-toxic. In one or more embodiments, the wetting agent 5 is an aqueous wetting agent. In one or more embodiments, the wetting agent 5 comprises water, a C1-6 alcohol, such as methanol, ethanol, propanol, isopropanol, or a combination thereof. The second compartment 22 contains at least one substrate 4. A breakable seal 30 is disposed between the first compartment 21 and the second compartment 22. As illustrated in FIG. 3, the breakable seal 30 is breakable to allow the wetting agent 5 to flow into the second compartment 22 through the aperture or rupture formed in response to the seal breaking and contact with the substrate 4 as indicated by arrow 6. The substrate 4 comprises liquid-absorbing materials and absorbs the wetting agent 5. The user then opens the flexible plastic container 2. More particularly, the user opens the second compartment 22, to remove the substrate 4, and uses the substrate 4 for the detection of a substance, such as the presence of an opioid. For example, the substrate 4 can be used for detecting the presence of an opioid on a surface when the surface is wiped or contacted with the substrate that has been wetted by the wetting agent 5 that moved through the breakable seal 30 in the direction of arrow 6.

Prolonged contact of the wetting agent 5 with the at least one substrate 4 may cause a reaction and decrease efficiency of the active ingredients in the substrate 4. Thus, it is desirable to keep them separated, via the breakable seal 30, until the time at which the user desired to determine or detect the presence of a substance, such as the opioid. As result, the detection effect of the kit 1 is maintained, and the shelf life of the kit 1 is extended by separating or segregating the wetting agent 5 from the substrate 4 until the time at which the kit is to be used to detect the presence of the substance or opioid.

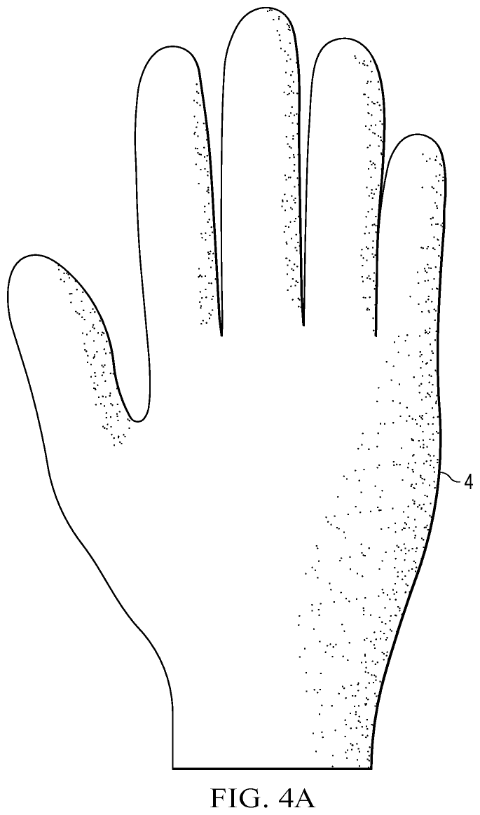

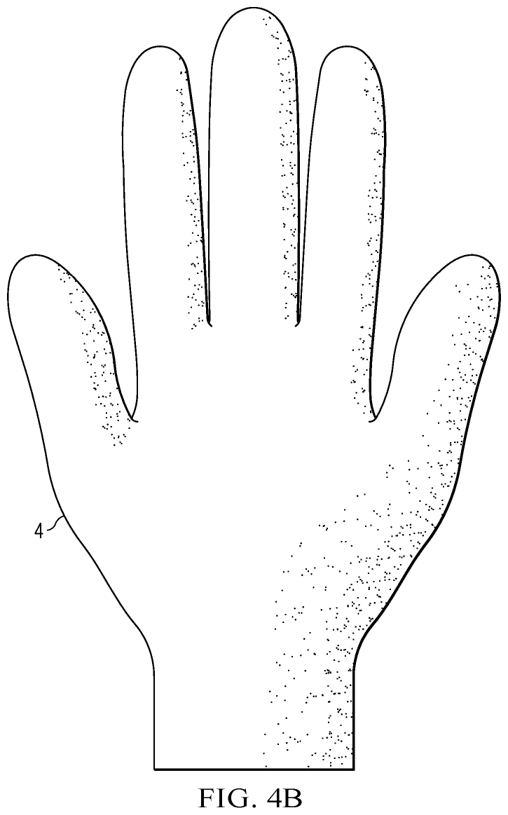

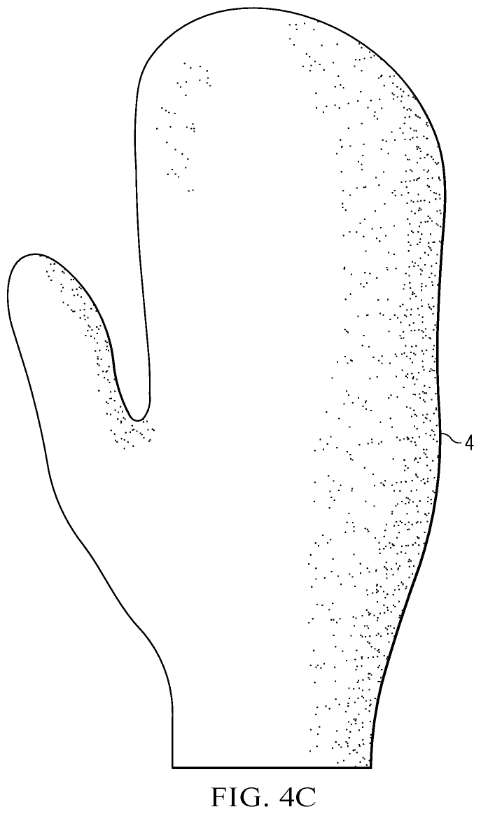

In some embodiments, such as shown in FIG. 4A-4C, the substrate 4 is formed into a glove or mitten, allowing the user to wear the substrate 4 on a hand while detecting the presence of the opioid. As such, the detection can be done by movements which the user is accustomed to, such as wiping or rubbing movements. Alternatively, the substrate can a generally rectangular shape, similar to that of a conventional napkin or “wipe.”

As illustrated in FIGS. 1-3, in some embodiments, the flexible plastic container 2 may comprise two pieces of plastic sheets. The first compartment 21 and the second compartment 22 are defined between two pieces of plastic sheets. The two pieces of plastic sheets are sealed with each other by, for example, adhesives or heat sealing, such that the first compartment 21 and the second compartment 22 are hermetically sealed compartments. In some embodiments, as illustrated in FIGS. 1-3, the first compartment 21 and the second compartment 22 are defined by frangible seals formed by two pieces of plastic sheets. The flexible plastic container 2 may comprise any flexible plastic material suitable for storing the wetting agent 5 fluid. For example, in some embodiments, the treating fluid is a hypochlorous acid solution, and the flexible plastic container 2 comprises LDPE, HDPE, polypropylene, PET, nylon, or combinations thereof. In some embodiments, the flexible plastic container 2 is made from metalized plastic sheet to prevent the decomposition of the active ingredients, such as hypochlorous acid, caused by light (UV radiation).

As illustrated in FIG. 1, the first compartment 21 and the second compartment 22 are formed as hermetically separate compartments. In some embodiments, the first compartment 21 and the second compartment 22 are separated by a sealing portion 3 formed across the flexible plastic container 2. The sealing portion 3 may be formed by, for example, adhesives or heat sealing. The sealing portion 3 may be formed as a frangible seal. The wetting agent 5 in the first compartment 21 is discussed in greater detail herein with respect to the type of fluid wetting agent and its interaction (when breakable seal 30 is broken) with the substrate 4 in the second compartment 22.

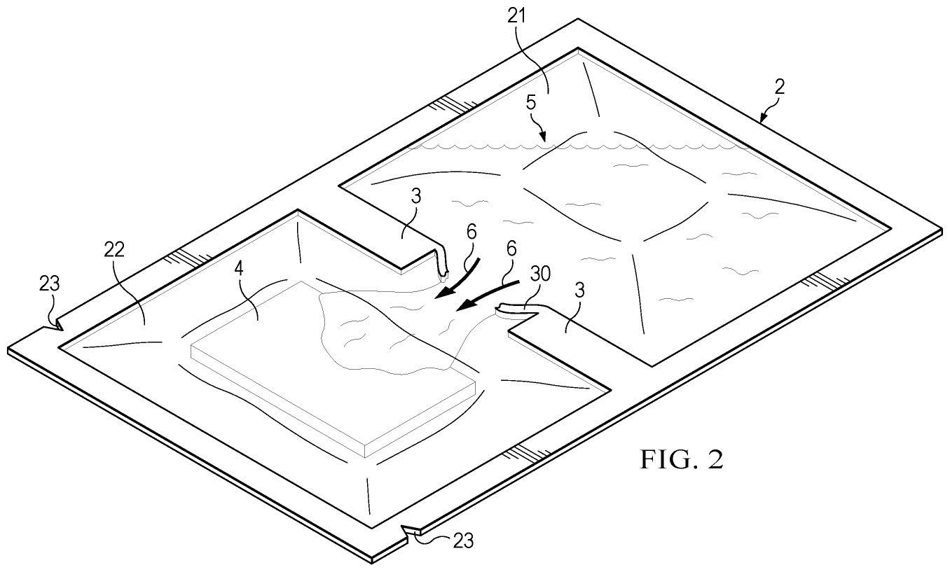

As illustrated in FIGS. 2-3, the second compartment 22 contains at least one substrate 4. In some embodiments, the second compartment 22 contains a pair of substrate 4 (i.e., two gloves or two mittens or two wipes). In other embodiments, the second compartment 22 contains more than two substrates 4 (i.e., three or more gloves, or three or more mittens, or three or more wipes). The substrate 4 may be a liquid-absorbing material that is formed into a shape, for example, cloth wipes, sponges, swabs, glove(s) or mitten(s). In some embodiments, the substrate 4 comprises non-woven fabric made from polymer fibers. The at least one substrate 4 may be compressed, for example by vacuum packaging, to reduce the volume. As illustrated in FIG. 4A-4C, in some embodiments, the substrate 4 is formed into a glove, such that the substrate 4 can be worn on hand by the user during the detection of the opioid or other substance. As illustrated in FIG. 4B, the substrate 4 may be formed into a glove having a symmetric shape, such that the user can conveniently switch between the faces of the substrate 4 on one hand, or alternative resulting in an ambidextrous glove that can be worn by either the left hand or the right hand. In some embodiments, the substrate 4 is formed into a glove with five finger pockets having identical shapes or differing shapes (for example a smaller thumb sleeve as compared to the four finger sleeves). As illustrated in FIG. 4C, the substrate 4 may be formed into a mitten glove. The substrate 4 may be formed into various shapes that can be worn or equipped on the hand, or otherwise being capable of being grasped by the hand (e.g., a wipe).

In some embodiments, the at least one substrate 4 is capable of absorbing at least some, a majority of, or substantially all of the wetting agent 5 from the first compartment 21. In the exemplary embodiment in which substantially all of the wetting agent is absorbed by the substrate, no liquid or substantially no liquid will spill out from the flexible plastic container 2 when the flexible plastic container 2 is opened by the user, and no liquid will drop or spill from the substrate 4 when the substrate 4 is used for the detection of a substance, such as an opioid. In some embodiments, the first compartment 21 contains an amount of the wetting agent that is a range from about one-half fluid ounce to about four fluid, and the second compartment 22 contains a pair of substrates 4 capable of absorbing that amount of fluid in total.

The breakable seal 30 is disposed between the first compartment 21 and the second compartment 22. The breakable seal 30 may be formed by, for example, adhesives or heat sealing. In some embodiments, as illustrated in FIGS. 1-2, the breakable seal 30 is formed as part of the sealing portion 3 formed across the flexible plastic container 2, and is narrower than other parts of the sealing portion 3. As such, the breakable seal 30 is the weakest part of the sealing portion 3, and is the part of the sealing portion 3 that breaks first when pressure is applied on the first compartment 21. The breakable seal 30 may have a length within a range of 0.5 cm to 5 cm. In some embodiments, the breakable seal 30 is formed across the whole width of the flexible plastic container 2, such that the breakable seal 30 comprises the entire sealing portion 3.

The breakable seal 30 is breakable by applying pressure to the first compartment 21 containing the wetting agent. In some embodiments, the breakable seal 30 is configured to break under a pressure caused by the user squeezing the first compartment 21, for example, a compressed fluid pressure against the frangible seal of the sealing portion 3. For example, the breakable seal 30 may be configured to break when the first compartment 21 is squeezed with a force within a range of 5 to 400 newtons, including 10 to 350 newtons, 50 to 350 newtons, and 100 to 300 newtons. In some embodiments, the breakable seal 30 is configured to break when the first compartment 21 is squeezed with a force of about 300 newtons.

As illustrated in FIGS. 1-2, the breakable seal 30 may have a wavy shape or a zigzag shape, in order to assist the breaking of the breakable seal 30. In some embodiments, the breakable seal 30 is breakable by tearing apart the sealing portion 3.

When the breakable seal 30 is broken, as illustrated in FIG. 2, the first compartment 21 and the second compartment 22 are open to, or in fluid communication with, each other. As such, the wetting agent 5 flows from the first compartment 21 into the second compartment 22 as indicated by arrow 6, and is absorbed by the at least one substrate 4. As illustrated in FIG. 3, the second compartment 22 of the flexible plastic container 2 can be opened, for example by tearing off one end, such that the user can remove the substrate 4 that has been wetted via the wetting gent and use it for the detection of a substance, such as an opioid.

The flexible plastic container 2 may be formed with one or more cutout 23. The cutout 23 may allow the user to tear open the flexible plastic container 2 and remove the substrate 4. Notches, slits, and/or perforations may additionally or alternatively be used to facilitate tearing.

Having thus described exemplary components of the packaging, reference is now made to the substrate, dyes, and other components that effectuate a color-change detection of a substance.

The self-contained substance detection kit 1 of the present disclosure provides a drug detection kit, and a method for rapid detection of opioids and other amine-containing drugs on a surface. The drug detection kit includes the substrate 4 which carries a dye in the second compartment 22 of the container 2.

Suitable substrates 4 provide a solid support for the dye, and may be selected from a wide variety of materials. The form of the substrate is not particularly limited. In one or more embodiments, the substrate 4 in the second compartment 22 of the drug detection kit may be formulated as a wipe, a swab, or a glove.

Exemplary substrates that are suitable include films, woven and nonwoven fabrics, cellulosic substrates such as tissues, paper towels, coform materials, airlaid materials, bonded-carded webs, and the like. Nonexclusive examples of substrates are described in U.S. Pat. Nos. 4,775,582 and 4,853,281, 4,833,003, 4,511,488, and the '446 Publication, all of which are incorporated herein by reference. In one or more embodiments, the substrate may be in the form of a wipe, a swab, a mitten, or a glove. In one or more embodiments, the substrate 4 may also be referred to as a mat.

In one or more embodiments, the substrate 4 that is packaged in the second compartment 22 is a coherent fibrous sheet. The fibrous sheets may comprise natural fibers (e.g. wood pulp, cotton, bamboo, hemp, etc.), synthetic fibers (e.g. polyolefin, polyester, polyamide, polylactic acid, rayon, lyocell, etc.) or combinations of natural and synthetic fibers.

In one or more embodiments, the substrate 4 comprises a polyester amide polymer having pendant functional groups, as described in U.S. Pat. No. 9,593,201, which is incorporated by reference herein. In one or more embodiments, the identity of the pendant groups in the polyester polymer influence the wetting nature of the substrate, which in turn influences the pick-up efficiency of the substrate. The pendant groups in the functional polyester may also influence interactions with the opioid analyte, and hence the pendant groups can be used to modulate reaction times and discriminate between different classes of analytes such as heroin and fentanyl. Advantageously, the functional polyester may be selected to favorably enhance the technology for binding of drug particles, modulation of response time, and color/tint of the substrate after contact with the opioid. By varying the pendant functional groups, the polymer fibers may be tailored for wettability, interactions with the silica particles and the response time of the color change.

In one or more embodiments, the substrate 4 may comprise an air-laid nonwoven web. Examples include meltblown, spunbond, and bonded-carded web materials. Air-laid nonwoven sheets, and methods of making the same, are described in U.S. Pat. Nos. 3,849,241, 4,340,563, 4,443,513, 4,548,856, 4,853,281, 5,382,400, 5,575,874, 6,224,977, 6,811,638, 6,946,413, and U.S. Pat. App. Pub. Nos. 2004/0192136 A1 and 2006/0008621 A1, all of which are incorporated herein by reference.

In one or more embodiments, the substrate 4 may comprise a coform nonwoven webs. Coform nonwoven webs may be formed by the comingling of polymeric fibers and absorbent fibers, such as polyolefin fibers and cellulosic fibers, as the fibers are entrained by a common airstream before they are deposited onto a forming surface. Coform sheet materials are described U.S. Pat. Nos. 4,100,324 and 5,350,624, and U.S. Pat. App. Pub. Nos. 2011/151596 A1, all of which are incorporated herein by reference. In one or more embodiments, the coform sheet may comprise a matrix of thermoplastic polymeric meltblown fibers and wood pulp fibers. Various suitable materials may be used to provide the polymeric meltblown fibers, such as, for example, polypropylene microfibers. In one or more embodiments, the polymeric meltblown fibers may be elastomeric fibers formed from elastomeric resins such as, for example, VISTAMAXX elastic olefin copolymer resin (available from ExxonMobil Corporation) or KRATON G styrene-ethylene/butylene-styrene and styrene-ethylene/propylene-styrene polymer resins (available from Kraton Performance Polymers, Inc.).

In one or more embodiments, the substrate 4 may comprise hydroentangled nonwoven sheet materials. Hydroentangled nonwoven webs are sometimes referred to as spunlace fabrics. In certain aspects, hydroentangling readily allows for the combination of different fiber types, such as combining fibers of distinct composition (e.g. polymeric fibers and wood pulp fibers) or fibers of different sizes. Hydroentangled materials, and methods of making the same, are described U.S. Pat. Nos. 3,485,706, 3,620,903, 5,009,747, 5,284,703, and 6,200,669, all of which are incorporated herein by reference.

In one or more embodiments, the substrate 4 may comprise a water dispersible and/or biodegradable sheet material. Examples of dispersible and/or degradable nonwoven fibrous materials are described in U.S. Pat. Nos. 5,667,635, 6,750,163, and 6,960,371, and U.S. Pat. App. Pub. No. 2017/0233912 A1, all of which are incorporated herein by reference.

In one or more embodiments, the substrate 4 of the drug detection kit 1 or device may further include one or more optional ingredients selected from, but not limited to, color enhancers, catalysts, mechanical property modifiers, optical brighteners, anti-static agents, flame retardants, lubricants, wetting agents, softeners, mordants, and inorganic additives. The optional ingredients may be embedded within, absorbed into, dispersed upon or within, or coated upon the substrate. Chemicals and additives that are typically employed in dyeing processes and methods may be employed with the drug detection device of the present invention, including one or more of soda ash fixer, urea, Dharma dye fixative, Synthrapol, Dharma professional textile detergent, Milsoft, sodium alginate, Superclear, Calsolene oil, Bleach-Stop, Dharma color remover, optic whitener, ammonium sulfate, Jacquard silk salt, Retayne, Ludigal F, Dharma discharge paste, alum, potassium alum, and Fiber etch.

In one or more embodiments, the drug detection kit 1 or device includes titanium dioxide. The titanium dioxide may advantageously function as an optical brightener in the drug detection device of the present invention.

Inorganic additives include zeolites, alumina and silica. Advantageously, in one or more embodiments, inorganic additives may be selected to increase the selectivity of the opioid detection device. In one or more embodiments, the substrate may be a composite of an inorganic-polymer matrix embedded with one or more chemicals to provide indication of the presence of opioids. In one or more embodiments, the substrate may be fabricated from polymers and silica particles.

Examples of mordants include aluminum acetate, titanium oxalate, alum, iron salts, copper salts, tin salts, siloxanes, acrylates, sodium chloride, chrome alum, tannins, vinegar, baking soda, tara powder, cream of tartar, washing soda, tannic acid, certain salts of aluminum, chromium, copper, iron, iodine, potassium, sodium, tungsten, and tin, and combinations of any of the above.

The substrate 4 should have sufficient mechanical strength and good processability, and should not be soluble in alcoholic or aqueous medium.

In one or more embodiments, the substrate 4 has a polarity that is sufficient to interact with the dye, such that they dye does not leach out during processing or use of the opioid detection device.

In one or more embodiments, the substrate 4 is characterized by a dry basis weight of from about 25 g/m2 to about 420 g/m2, in other embodiments, from about 30 g/m2 to about 300 g/m2, in other embodiments, from about 35 g/m2 to about 250 g/m2, in other embodiments, from about 40 g/m2 to about 200 g/m2, in other embodiments, from about 45 g/m2 to about 150 g/m2, and in still further embodiments, from about 50 g/m2 to about 125 g/m2.

In one or more embodiments, the substrate 4 may be a porous substrate, where porous refers to the ability of the substrate to absorb liquid. The porous structure is advantageous because: (1) it allows for efficient encapsulation of dyes that are responsible for the color change response upon contact with the opioid, (2) the retention of moisture is more efficient and (3) higher adsorption of the opioid particles to the mats.

In one or more embodiments, the substrate 4 may be a multi-layer substrate, where at least one layer of the substrate is porous.

In other embodiments, the substrate 4 is non-porous and impermeable to the amine-containing drugs and other harmful substances. For example, when the drug detection device is a glove, a non-porous and impermeable substrate is advantageous because it protects the user from direct contact with potentially harmful substances.

If desired, the substrate 4 may be further treated by one or more techniques as is known in the art to improve the durability, strength, hand, aesthetics, texture, and/or other properties of the substrate material. For instance, a nonwoven web may be pattern bonded or embossed by the use of heat, pressure and/or ultrasonic energy. The nonwoven substrate materials may be bonded by continuous and/or discontinuous lines, by patterns of numerous discrete elements, or other patterns as may be desired. In one or more embodiments, the nonwoven web may be bonded along the periphery of the sheet or simply across the width or cross direction of the web adjacent to the edges.

In these or other embodiments, a resin, latex or adhesive may be applied to the substrate 4 by, for example, spraying or printing, to achieve the desired nature and degree of bonding. In one or more embodiments, the substrate may be treated by various other known techniques such as, for example, stretching, needling, creping, printing, dyeing, and so forth.

In one or more embodiments, the fibrous sheets may be used to form laminates with one or more additional sheet materials.

The drug detection device 1 or kit of the present disclosure may further comprise a dye on the substrate 4. In one or more embodiments, the dye enables detection of the presence of one or more opioids and other amine-containing drugs. In one or more embodiments, the dye enables differentiation between one or more opioids and other amine-containing drugs. That is, in one or more embodiments, the dye enables the identification of a specific drug. In one or more embodiments, the dye undergoes a color change in the presence of an opioid or other amine-containing drug. Advantageously, the color change is rapid and easily visible to the human eye. In one or more embodiments, the color change occurs in less than 1 minute, in other embodiments, in less than 30 seconds and in other embodiments, in less than 3 seconds. In one or more embodiments, the color change is virtually instantaneous.

In one or more embodiments, the dye is selected from the group comprising or consisting of malachite green oxalate, brilliant green, eosin yellowish, erythrosine b, methyl green, methyl violet, picric acid, crystal violet, bromocresol green, m-cresol purple, thymol blue, p-xylenol blue, cresol red, eosin bluish, quinaldine red, 2,4-dinitro phenol, 4-(dimethylamino) azobenzol, bromochlorophenol blue, bromophenol blue, congo red, methyl orange, 1-naphtholphthalein, m-cresol purple, thymol blue, p-xylenol blue, phenolphthalein, thymolphthalein, alkali blue, alizarin yellow gg, indigo carmine, epsilon blue, 2,5-dinitrophenol, alizarin sulphonic acid, methyl red, chlorophenol red, litmus, bromocresol purple, bromophenol red, 4-nitrophenol, bromoxylenol blue, bromothymol blue, phenol red, 3-nitrophenol, neutral red, and titan yellow.

Regarding dye preparation, the desired dye may be coated onto the substrate 4 (prior to assembly of kit 1 and being installed in the second compartment 22) in any of the commonly used methods for coating substrates, such as dip and nip, spraying, ink jet printing, etc. The dye may also be physically adsorbed or covalently conjugated onto latex, silica, cellulosic fibers or other polymeric fibers, which may be placed on or in the substrate generally or in a pattern.

Regarding pre-treatment, in one or more embodiments, the substrate 4 may be pre-treated prior to applying the dye pre-mix (and prior to assembly/manufacture and installation into the second compartment 22). Any of the optional ingredients discussed above for the substrate may be applied in a pre-treatment step.

Regarding dye pre-mix, in one or more embodiments, a dye pre-mix is prepared by dissolving or suspending the dye in a solvent. Suitable solvents include water and aqueous-based solvents, C1-6 alcohols, glycols, and combinations thereof. In one or more embodiments, the solvent comprises water, ethanol, methanol, or a combination thereof.

In one or more embodiments, the dye pre-mix may further include one or more optional ingredients selected from, but not limited to, color enhancers, catalysts, mechanical property modifiers, optical brighteners, anti-static agents, flame retardants, lubricants, wetting agents, softeners, mordants, and inorganic additives. In one or more embodiments, one or more optional ingredients may be selected to provide a desired pH or ionic strength.

Regarding dry substrate preparation, in one or more embodiments, the substrate 4 and dye is prepared (prior to assembly of kit 1 and being installed in the second compartment 22) by a method that includes the steps of (1) preparing a dye pre-mix by dissolving or mixing a dye with a solvent; (2) applying the dye pre-mix to a substrate; (3) removing the solvent. In one or more embodiments, the solvent may be removed through evaporation. In one or more embodiments, the step of removing the solvent may be accomplished with heating. In one or more embodiments, the step of removing the solvent may be accomplished in a vacuum chamber. In one or more embodiments, the step of removing the solvent may be accomplished at room temperature and pressure.

Regarding wet substrate preparation, in other embodiments, the substrate 4 and dye is prepared (prior to assembly of kit 1 and being installed in the second compartment 22) by a method that includes the first two steps of the above method, namely, (1) preparing a dye pre-mix by dissolving or mixing a dye with a solvent; and (2) applying the dye pre-mix to a substrate. However, rather than removing the solvent to form a dry device, at least some of the liquid is left on the device, such that the device is moist. The moist device may then be enclosed in an container 2 of the airtight package or kit 1 for storage and/or shipment. The package may be opened before use.

In one or more embodiments, the amount of dye is at least an amount that is sufficient to produce a change in color upon contact with an opioid or other desired substance that is to be detected, where the color change is detectable to the unaided eye. In one or more embodiments, the amount of dye is at least about 0.01, in other embodiments, at least about 0.05, and in other embodiments, at least about 0.1 wt. %, based upon the total weight of the substrate and dye, on a dry basis. In one or more embodiments, the amount of dye is from about 0.01 to about 10 wt. %, in other embodiments, from about 0.05 to about 5, and in other embodiments, from about 0.1 to about 3 wt. %, based upon the total weight of the substrate and dye, on a dry basis.

In one or more embodiments, the substrate 4 may be post-treated after applying the dye pre-mix (and prior to assembly of kit 1 and being installed in the second compartment 22). Any of the optional ingredients discussed above for the substrate may be applied in a post-treatment step.

In one or more embodiments of either of the above dry and wet methods of preparation, a subsequent washing step may follow the step of applying the dye pre-mix to the substrate 4. In one or more embodiments, the dye may be fixed onto the substrate by washing the dye/substrate with a fixative, detergent, wetting agent, surfactant, or combination thereof. Examples of fixatives include detergents such as Synthrapol SP and Retayne.

Container 2 is assembled after the substrate 4 and dye have been prepared in the manner described above. The substrate 4 is placed in the second compartment 22 and the wetting agent 5 is placed into the first compartment 21. The method of manufacture may be effectuated by any known manner which disposes these components in these locations with the breakable seal 30 therebetween.

In operation, the user breaks the breakable seal 30 by applying pressure to the first compartment 21 by, for example, squeezing the first compartment 21. The user may also break the breakable seal 30 by tearing apart the sealing portion 3. As result of rupturing seal 30, the first compartment 21 and the second compartment 22 are in fluid communication with each other. The wetting agent 5 contained in the first compartment 21 flows into the second compartment 22 as indicated by arrow 6 and is absorbed by the at least one substrate 4 (or otherwise contacts the substrate 4) contained in the second compartment 22, wherein the substrate 4 has been prepared in the manner described above. The user opens the flexible plastic container 2, and more particularly the second compartment 22, removes the substrate 4, and uses the substrate 4 for the detection of the substance or opioid. In some embodiments, the substrate 4 is formed into a glove, and the user wears the substrate 4 on a hand during the detection of the substance or opioid. In other embodiments, the substrate is formed as a wipe or sheet, and the user grasps the substrate with the hand and wipes the surface to detect the substance or opioid by viewing a color change of the substrate 4 when the opioid is present.

With continued reference to the operation of the kit 1 of the present disclosure, the drug detection device kit of the present invention is useful to indicate the presence of an opioid or other amine-containing drug, i.e. a target analyte. Thus, the present disclosure also provides a method for detecting a target analyte on a suspected surface. After breaking the breakable seal 30 to apply the wetting agent 5 to the substrate and removing the wetted substrate 4 from the container, the method includes the steps of contacting a surface that is suspected of containing a target analyte with a drug detection device that includes substrate 4 carrying a dye that is capable of producing a visible color change to at least a portion of the substrate 4 upon exposure to the target analyte. The target analytes, substrates, dyes, and color changes are as described herein.

In one or more embodiments, applying the wetting agent 5 to either or both of the substrate 4 and the suspected surface prior to contact facilitates the reaction of the target analyte and the dye, and thus speeds up and/or enhances the color change. In one or more embodiments, the method of the present disclosure includes the step of applying the wetting agent 5 to the suspected surface or the substrate 4 prior to the step of contacting the surface with the drug detection device. In these or other embodiments, the container 2 includes the wetting agent 5, and is provided in a sealed, airtight package or container. The method of the present disclosure may further include the step of removing the substrate 4 from the airtight package prior to use. In one or more embodiments, the wetting agent 5 is non-toxic. In one or more embodiments, the wetting agent 5 is an aqueous wetting agent. In one or more embodiments, the wetting agent comprises water, a C1-6 alcohol, such as methanol, ethanol, propanol, isopropanol, or a combination thereof.

In one or more embodiments, the substrate 4 of drug detection device may be in the nature of a wipe, sheet, mat, swab, mitten or glove. As further described herein, a user may simply bring the substrate 4 of the drug device into contact with a suspected surface. In one or more embodiments, all of the surfaces of the substrate may be uniform in terms of the ability of the surface to change color. In these embodiments, any portion of the substrate may be employed in the method of use. In other embodiments, one or more portion of the surfaces of the substrate may be configured to change color. The portion of the substrate that is configured to change color upon contact with an opioid or other amine-containing drug may be referred to as the color-changing portion. For purposes of this specification, reference to a change of color for the drug detection device should be interpreted to mean a change of color for at least the color-changing portion of the device.

If a target analyte, i.e. an opioid or other amine-containing drug, is present on the suspected surface, the detection device, or at least the color-changing portion of the device, will change color.

In one or more embodiments, the drug detection device may originally be blue color, and upon contact with a target analyte, the target analyte reacts with the dye and the detection device becomes white. In one or more embodiments, contact with a target analyte results in a color change from orange to green. In one or more embodiments, contact with a target analyte results in a color change from orange to blue. In one or more embodiments, contact with a target analyte results in a color change from blood red to yellow. In one or more embodiments, contact with a target analyte results in a color change from blood red to white. In one or more embodiments, contact with a target analyte results in a color change from red to blood red. In one or more embodiments, contact with a target analyte results in a color change from white or off-white to blue.

Advantageously, the change in color may be virtually instantaneous, and does not require any additional heating, or addition of acids or bases. In one or more embodiments, the color change is readily visible and can be discerned by the human eye.

Target analytes (the substance to be detected by the drug detector device) that may be detected by the drug detection devices and methods of the present invention include opioids and other amine-containing drugs. Many of these substances are used medically for pain relief, can be addictive, and are controlled substances. Overexposure may result in a fatal overdose. Examples include opiates, which refers to drugs that can be derived from opium. Specific examples of opioids and other amine-containing drugs include morphine, semi-synthetic and synthetic drugs such as hydrocodone, oxycodone, fentanyl, carfentanyl, acetyl fentanyl, tetrahydrofuran fentanyl, cocaine, methamphetamine and their analogues, as well as drug antagonists such as naloxone, and endogenous peptides such as the endorphins. In one or more embodiments, the target analyte includes heroin, fentanyl, oxycodone (OxyContin®), hydrocodone (Vicodin®), codeine, morphine, or a mixture or derivative thereof. In one or more embodiments, the target analyte includes one or more tertiary amine groups.

Advantageously, one or more embodiments of the present disclosure provide a safe and reliable way for first responders to have a single kit that they can carry as a self-contained drug detection kit 1. The kit allows the first responder to rupture the breakable seal 30 to wet the substrate with the wetting agent. Then, the user (e.g., the first responder) may open the package and remove the wetted substrate 4. When the substrate is embodied as a glove or a mitten, the substrate may be donned by the user's hand. If the substrate 4 is a simple wipe, the user may simply grasp the wipe with their hand. Then the substrate 4 may be wiped on a surface to detect the presence of opioids and other amine-containing drugs, thus providing a warning to avoid inadvertent exposure to potentially lethal drugs. The drug detection methods of the present disclosure are cost-effective and easy-to-use. In certain embodiments, where the drug detection device is a wipe or glove, large surface areas may be tested, such as table tops, cars, prison cells, etc. In other embodiments, the substrate 4 of the drug detection device or kit 1 is a swab that is adapted for hard to reach areas such as car consoles, pant pockets, etc. Microgram amounts of the target analyte can be detected. The drug detection device or kit 1 may be easily and safely carried in a patrol car, an ambulance or on a person.

Unless explicitly stated that a particular shape or configuration of a component is mandatory. Any of the elements, components, or structures discussed herein may take the form of any shape. Thus, although the figures depict the various elements, components or structures of the present disclosure according to one or more exemplary embodiments, it is to be understood that any other geometric configuration of that element, component or structure is entirely possible. For example, instead of the container 2 being rectangular in configuration, the container 2 (or each respective compartment 21, 22) can be circular, semi-circular, triangular, square, pentagonal, hexagonal, heptagonal, octagonal, decagonal, dodecagonal, diamond shaped or another parallelogram, trapezoidal, star-shaped, oval, ovoid, lines or lined, teardrop-shaped, cross-shaped, donut-shaped, heart-shaped, arrow-shaped, crescent-shaped, any letter shape (i.e., A-shaped, B-shaped, C-shaped, D-shaped, E-shaped, F-shaped, G-shaped, H-shaped, I-shaped, J-shaped, K-shaped, L-shaped, M-shaped, N-shaped, O-shaped, P-shaped, Q-shaped, R-shaped, S-shaped, T-shaped, U-shaped, V-shaped, W-shaped, X-shaped, Y-shaped, or Z-shaped), or any other type of regular or irregular, symmetrical or asymmetrical configuration. Furthermore, when the substrate 4 is a wipe, instead of the wipe being rectangular in shape, it may take on any the aforementioned shapes or configurations.

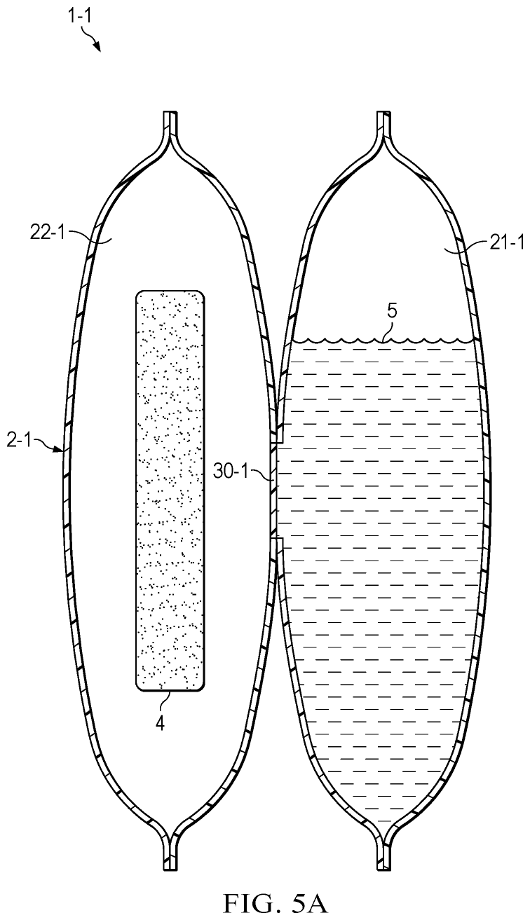

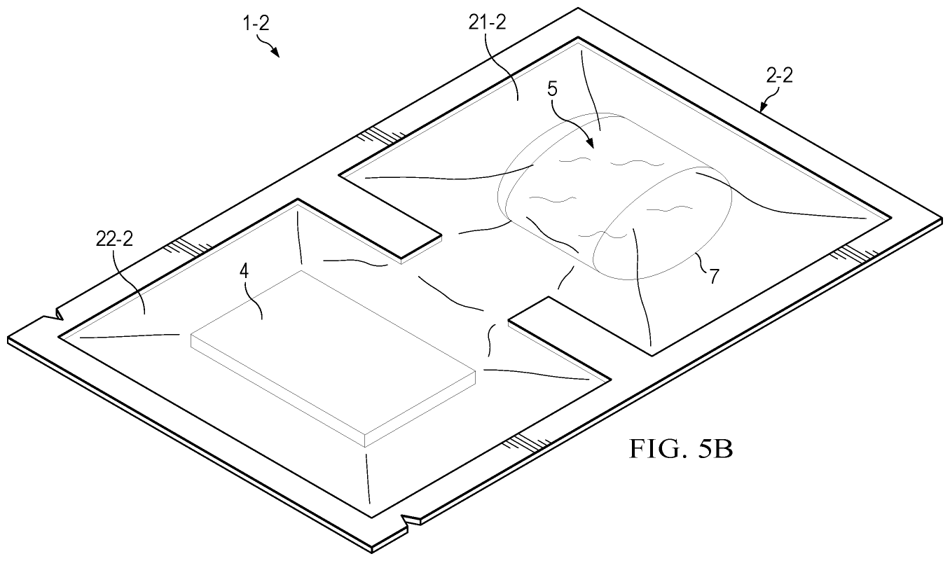

FIG. 5A depicts an alternative embodiment of kit 1-1 having a container 2-1 with a first compartment 21-1 and a second compartment 22-1. Rather than one compartment being located vertically above the other (as shown previously), container 2-1 arranges the first compartment 21-1 and second compartment 22-1 in a front-to-back relationship with the breakable seal 30-1 therebetween. The wetting agent 5 is located in the first compartment 21-1 and may be transferred to the second compartment 22-1 in response to the rupture of breakable seal 30-1. Then, the substrate 4 may be removed from the second compartment 22-1 and used in the manner taught herein.

FIG. 5B depicts an alternative embodiment of kit 1-2 having a container 2-2 with a first compartment 21-2 and a second compartment 22-2. Rather than having a breakable seal between the two compartments, the compartments are in open fluid communication through the aperture or opening therebetween. Since there is no breakable seal between the compartments, the wetting agent 5 may be contained in a breakable vial 7 located in the first compartment 21-2. The vial 7 is configured to break in response to a pressure being applied to the vial. The breaking of vial 7 cause the wetting agent 5 to flow through the aperture or opening to thereby wet the substrate 4. Then, the substrate 4 may be removed from the second compartment 22-2 and used in the manner taught herein to detect the presence of a substance, such as an opioid, on a surface.

Other configurations of a container having two separated or segregated compartments, wherein one compartment retains the wetting agent 5 and the other compartment retains the substrate 4 that is configured to be wetted in response to a rupture, tear or deformation of the compartment retaining the wetting agent 5 are taught in U.S. Pat. No. 5,046,608, the entirety of which is incorporated herein by reference.

In other embodiments of the present disclosure, a kit or substrate 4 can be provided that is imbedded with chemical compounds that can detect the presence of the target analyte, such as an opioid, without the need to use the wetting agent 5 or wetting reagent 5. The wipe or substrate 4 may be manufactured by incorporating indicator compounds directly into the wipe material. These indicators are carefully chosen to undergo a noticeable color change upon contact with opioids or another target analyte. In this example, indicator compounds that exhibit a visible color change in the presence of opioids are chosen. These indicators are designed to selectively react with opioids while remaining stable and compatible with the wipe material. The selected indicator compounds are impregnated or coated onto the wipe material during its production. This ensures a uniform distribution of the indicators throughout the wipe. The color-changing wipe or substrate 4 is used to wipe the surface suspected of containing opioids. As the wipe comes into contact with the surface, any opioids present interact with the embedded indicators. If opioids (or another target analyte) are present, they react with the indicator compounds within the wipe material. This reaction triggers a chemical change in the indicators, resulting in a visible color change within the wipe. The color change serves as a clear indication of the presence of opioids on the surface. The color-changing wipe or substrate 4 is observed visually, and the resulting color change is compared to a reference scale or chart that correlates the intensity of the color change to the presence or concentration of opioids.

In this exemplary embodiment that does not use a wetting agent 5 or reagent 5, several indicator compounds can be utilized to detect the presence of opioids. The choice of indicator compounds depends on the specific opioid targeted for detection. For example, certain dyes can exhibit a color change in the presence of opioids. For instance, methylene blue has been employed to detect opioids like fentanyl and its analogs. It undergoes a color shift when it interacts with opioids, providing a visual indication of their presence. When opioids are present, they interact with methylene blue, causing a chemical reaction or complex formation that leads to a visible color shift. The color change can be easily observed and indicates the presence of opioids on the tested surface. Dye-based indicators are often selected based on their specific interactions with opioids and their ability to produce a distinct and easily distinguishable color change.

Additionally, in the embodiment in which the wetting agent is not present, fluorescent compounds can be used to detect opioids through fluorescence-based assays. These indicators emit light of specific wavelengths when excited by an appropriate light source. The presence of opioids can alter the fluorescence properties of these indicators, resulting in a change in the emitted light. These indicators consist of fluorescent compounds that emit light at specific wavelengths when excited by an appropriate light source. In the presence of opioids, the interaction can cause changes in the fluorescent properties of the indicators. These changes can include alterations in the intensity, wavelength, or lifetime of the emitted light. By detecting these changes, either visually or with specialized equipment, the presence of opioids can be identified. Fluorescent indicators offer the advantage of high sensitivity and the potential for quantitative analysis.

Further, in the embodiment in which the wetting agent is not present, chemosensors can be specific chemical compounds designed to detect and respond to the presence of opioids. They exhibit structural changes or alterations in their optical properties upon interacting with opioids. These changes can be used to signal the presence of opioids. Chemosensors are often tailored to bind selectively to specific opioid molecules, enhancing their sensitivity and selectivity. The structural or optical changes can manifest as color shifts, fluorescence changes, or variations in electrical conductivity, depending on the design of the chemosensor. Chemosensors can be synthesized using various strategies, including molecular recognition, supramolecular chemistry, and functionalized polymers, enabling the development of highly specific and sensitive detectors for opioids.

Still further, in the embodiment in which the wetting agent is not present, nanostructured indicators involve the use of nanomaterials, such as nanoparticles or quantum dots, to detect opioids. These nanostructures can be functionalized with specific receptors or ligands that have high affinity for opioids. When opioids come into contact with the nanostructured indicators, they bind to the receptors, resulting in changes in the optical properties of the nanoparticles. These changes can include alterations in absorbance, scattering, or surface plasm on resonance, leading to a detectable color change or shifts in other optical signals. Nanostructured indicators offer advantages such as enhanced sensitivity, tunable properties, and potential multiplexing capabilities.

Various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

While components of the present disclosure are described herein in relation to each other, it is possible for one of the components disclosed herein to include inventive subject matter, if claimed alone or used alone. In keeping with the above example, if the disclosed embodiments teach the features of A and B, then there may be inventive subject matter in the combination of A and B, A alone, or B alone, unless otherwise stated herein.

As used herein in the specification and in the claims, the term “effecting” or a phrase or claim element beginning with the term “effecting” should be understood to mean to cause something to happen or to bring something about. For example, effecting an event to occur may be caused by actions of a first party even though a second party actually performed the event or had the event occur to the second party. Stated otherwise, effecting refers to one party giving another party the tools, objects, or resources to cause an event to occur. Thus, in this example a claim element of “effecting an event to occur” would mean that a first party is giving a second party the tools or resources needed for the second party to perform the event, however the affirmative single action is the responsibility of the first party to provide the tools or resources to cause said event to occur.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

An embodiment is an implementation or example of the present disclosure. Reference in the specification to “an embodiment,” “one embodiment,” “some embodiments,” “one particular embodiment,” “an exemplary embodiment,” or “other embodiments,” or the like, means that a particular feature, structure, or characteristic described in connection with the embodiments is included in at least some embodiments, but not necessarily all embodiments, of the invention. The various appearances “an embodiment,” “one embodiment,” “some embodiments,” “one particular embodiment,” “an exemplary embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, the method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

To the extent that the present disclosure has utilized the term “invention” in various titles or sections of this specification, this term was included as required by the formatting requirements of word document submissions pursuant the guidelines/requirements of the United States Patent and Trademark Office and shall not, in any manner, be considered a disavowal of any subject matter.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed is:

1. A self-contained substance detection kit comprising:
a container defining a first compartment and a second compartment;
a wetting agent in the first compartment, wherein the wetting agent is an aqueous wetting agent;
at least one substrate in the second compartment, wherein the at least one substrate carries a dye that produces a visible color change upon exposure to a target analyte; and
a breakable seal between the first compartment and the second compartment, wherein the breakable seal is breakable in response to applying pressure to the first compartment, wherein when the breakable seal is broken, an aperture is defined that establishes open fluid communication between the first compartment and the second compartment to allow the wetting agent to flow into the second compartment and wet the at least one substrate.

2. The self-contained substance detection kit of claim 1, wherein the dye is selected from the group consisting of malachite green oxalate, brilliant green, eosin yellowish, erythrosine b, methyl green, methyl violet, picric acid, cresol red, crystal violet, m-cresol purple, thymol blue, bromocresol green, cresol red, p-xylenol blue, eosin bluish, quinaldine red, 2,4-dinitro phenol, 4-(dimethylamino) azobenzol, bromochlorophenol blue, bromophenol blue, congo red, methyl orange, 1-naphtholphthalein, m-cresol purple, thymol blue, p-xylenol blue, phenolphthalein, thymolphthalein, alkali blue, alizarin yellow gg, indigo carmine, epsilon blue, and titan yellow.

3. The self-contained substance detection kit of claim 1, wherein the target analyte is selected from the group consisting of opioids or amine-containing drugs.

4. The self-contained substance detection kit of claim 1, wherein the target analyte is selected from the group comprising of morphine, hydrocodone, oxycodone, fentanyl, carfentanyl, naloxone, endorphins, heroin, codeine, cocaine, and mixtures and derivatives thereof.

5. The self-contained substance detection kit of claim 1, wherein the substrate comprises a film, a woven fabric, a nonwoven fabric, a cellulose tissue, a paper towel, a coform material, an airlaid material, a polyester amide polymer having pendant functional groups, or a bonded-carded web.

6. The self-contained substance detection kit of claim 1, wherein the substrate comprises natural fibers, synthetic fibers, or combinations thereof.

7. The self-contained substance detection kit of claim 1, wherein the substrate comprises fibers selected from the group consisting of wood pulp, cotton, bamboo, hemp, polyolefin, polyester, polyamide, polylactic acid, rayon, lyocell, and combinations thereof.

8. The self-contained substance detection kit of claim 1, wherein the substrate is formed as one of a wipe, a swab, a glove and a mitten.

9. The self-contained substance detection kit of claim 1, further comprising:
a color enhancer; and
a catalyst.

10. The self-contained substance detection kit of claim 1, wherein the breakable seal is formed from the same material as the material that defines the first compartment and the second compartment.

11. The self-contained substance detection kit of claim 1, wherein the substrate and wetting agent are hermetically sealed in the container.

12. The self-contained substance detection kit of claim 1, wherein the aqueous wetting agent comprises at least one of water and a C1-6 alcohol.

13. The self-contained substance detection kit of claim 1, wherein the at least one substrate defines a glove within the second compartment.

14. The self-contained substance detection kit of claim 1, wherein the glove is symmetric to thereby define an ambidextrous glove.

15. A method for detecting the presence of a substance, the method comprising:
carrying a self-contained substance detection kit including a container defining a first compartment and a second compartment, the first compartment containing a wetting agent, wherein the wetting agent is an aqueous wetting agent, and the second compartment containing at least one substrate, wherein the at least one substrate carries a dye that produces a visible color change upon exposure to an opioid or other amine-containing drug;
breaking a breakable seal disposed between the first compartment and the second compartment, such that the wetting agent flows into the second compartment;
wetting the at least one substrate;
opening at least the second compartment to remove the at least one substrate;
contacting the at least one substrate with a surface; and
determining whether the dye has changed color, wherein a change in color is indicative of a presence of the opioid or other amine-containing drug.

16. The method of claim 15, further comprising:
after the step of contacting the at least one substrate with the surface, visually inspecting the at least one substrate to determine if any portion of the at least one substrate has changed color.

17. The method of claim 15, wherein at least a portion of the drug detection device undergoes a visible color change within about 30 seconds of contact with a target analyte.

18. The method of claim 15, wherein at least a portion of the drug detection device undergoes a visible color change within about 10 seconds of contact with a target analyte.

19. The method of claim 15, wherein at least a portion of the drug detection device undergoes a visible color change within about 5 seconds of contact with a target analyte.

20. The method of claim 15, wherein the dye is selected from the group consisting of malachite green oxalate, brilliant green, eosin yellowish, erythrosine b, methyl green, methyl violet, picric acid, cresol red, crystal violet, m-cresol purple, thymol blue, bromocresol green, cresol red, p-xylenol blue, eosin bluish, quinaldine red, 2,4-dinitro phenol, 4-(dimethylamino) azobenzol, bromochlorophenol blue, bromophenol blue, congo red, methyl orange, 1-naphtholphthalein, m-cresol purple, thymol blue, p-xylenol blue, phenolphthalein, thymolphthalein, alkali blue, alizarin yellow gg, indigo carmine, epsilon blue, and titan yellow.
